(19) United States
(12) Patent Application Publication
(10) Pub. No.: US 2009/0232853 A1
(43) Pub. Date: Sep. 17, 2009

(54) TREATMENT OF LAMINITIS

(76) Inventor: Patricia Ann Harris, Leicestershire (GB)

Correspondence Address: FULBRIGHT & JAWORSKI, LLP, 1301 MCKINNEY, SUITE 5100, HOUSTON, TX, 77010-3095, US

(21) Appl. No.: 11/909,245

(22) PCT Filed: Mar. 21, 2006
(86) PCT No.: PCT/GB2006/001028

§ 371 Date: Oct. 31, 2008

(30) Foreign Application Priority Data
Mar. 21, 2005 (GB) 0505756.7

(51) Int. Cl. A61K 9/00; A61K 36/906; A61K 31/12; A61K 31/137; A61K 31/352; A61K 38/47

(52) U.S. Cl. 424/400; 424/756; 514/685; 514/654; 514/456; 424/94.61

(57) ABSTRACT

The present invention provides a composition comprising one or more of a matrix metalloprotease inhibitor, a de-carboxylase inhibitor, a fructanase enzyme and/or a flavonoid for use in the prevention of laminitis

The present invention provides a composition comprising one or more of a matrix metalloprotease inhibitor, a decarboxylase inhibitor, a fructanase enzyme and/or a flavonoid for use in the prevention of laminitis. The present invention further provides a method for manufacturing the composition of the invention. In addition the present invention relates to the use of one or more of a matrix metalloprotease inhibitor, a decarboxylase inhibitor, a fructanase enzyme and/or a flavonoid in the manufacture of a composition for the prevention of laminitis and a method of preventing laminitis comprising administering a composition of the invention to an animal in need thereof.

Laminitis is one of the most important equine diseases worldwide in terms of animal suffering, mortality, lose of use and financial cost to the owner. Although many animals recover after an acute episode, many are affected for the rest of their lives resulting in loss of use to the owner and chronic or recurrent pain for the animal. A study in the UK estimated that there is a mortality rate of around 7.5% of affected animals and that around 3% of the UK horse population are chronically laminitic.

Laminitis literally means inflammation of the laminae which connect the pedal bone to the hoof wall. The dermal laminae interdigitate with the epidermal laminae to suspend the foot in the hoof. The force created by the animal's weight is passed down through the bones of the leg to the pedal bone. The laminae help transfer and dissipate the force from the bone to the hoof wall. In severe cases the condition may weaken the bond between the hoof wall and the pedal bone. This may cause the pedal bone to sink (‘sinker’) or rotate and drop (‘founder’) due to the upward pull of the ligaments.

A horse with laminitis is likely to be in pain and discomfort. When severe, horses may need to be euthanased. Once significant changes to the laminae have occurred, they are often irreversible. Even after successful treatment a horse may become more likely to suffer from recurring attacks.

The actual pathological process of laminitis is thought to be one of inflammation followed by degeneration and separation of the laminae. There are currently three main theories as to the pathophysiology of how laminitis develops, none of which are mutually exclusive and each results in the loss of functional integrity of the laminae which in turn leads to the observed clinical signs:—
Ischaemia—vasoconstriction potentially followed by free radical induced damage following reperfusion (and vasodilation during the acute stages).
Primary basement membrane pathology:—damage to the membrane that provides the link between the dermal and epidermal laminae—most likely involved with increased matrix-metallo-proteinase activity. This could be primary or secondary to the ischaemic-reperfusion.
Interference with epidermal cell structure and function.

Laminitis is believed to occur secondarily to a number of conditions including certain gastrointestinal diseases, in particular those which result in an increase in the permeability of the GI tract, such as colic. Certain infectious and toxic conditions (for example endometritis) and certain endocrine disorders (for example hyperadrenocorticism) are also associated with laminitis. In addition, laminitis may occur following adverse mechanical influences on the foot including excess weight bearing or excess foot trimming. The most common predisposing factors for laminitis are however, believed to be hyperadrenocorticism and excessive carbohydrate (such as starch, sugars, fructans etc) reaching the hindgut and being fermented. There is now an increasing awareness of the role of glucose and insulin resistance in laminitis.

Horses are non-ruminant herbivores that have evolved to subsist principally on a diet of fibrous vegetation, much of which can not be broken down by mammalian enzymes. Digestion of this fibrous material is principally carried out in the caecum and colon by a large population of microorganisms, primarily bacteria but also including protozoa, yeasts and fungi. These microorganisms are responsible for the digestion of fibrous material allowing the horse to use this material as a source of energy.

Wild and feral equids are thought to suffer far less from dietary induced metabolic disorders because they subsist on a diet that their digestive system has evolved to cope with, although they may suffer if they get access to inappropriate feeds and pastures. Metabolic disorders are much more frequently seen in domestic animals due to the addition of less fibrous foods to their diet, most noticeably cereal and access to starch/sugar/fructan rich pastures.

Access to large amounts of cereals, lush grass, other pasture species (such as legumes or clover or herbacious weeds or stemmy ‘stressed’ grass) or forage with a high sugar/starch or fructan content are the commonest recognised feed related predisposing causes of laminitis especially in ponies. Sugar is produced as an energy source in plants, including cereals and grasses, via photosynthesis. Depending upon the plant's energy requirements, this sugar is either metabolised directly or stored as storage carbohydrates for use at a later time. The main storage carbohydrate for cereals is starch (chains of glucose units linked together). Some pasture species may also contain starch as the main storage carbohydrate, such as clover which can have starch content of up to 50%. However, for some grass species, starch may account for as little as 10-15% of the total storage carbohydrate with the remainder being present as simple sugars such as sucrose or more complex molecules such as fructans. Fructans are polymers of fructose and can form between 5 and 50% of the dry matter of grass. Unlike starch and other simple sugars, fructans are believed to be effectively non-hydrolysable (i.e. not broken down by mammalian enzymes) and pass to a greater extent unmodified to the hindgut, where they act in a similar manner to a starch overload. A typical managed pasture may contain several types of grasses, clover (or other legume) and various herby weed species, which may therefore present a risk to ponies predisposed to laminitis from an excess of starch and/or fructan. It should be appreciated that in addition to the amount of fructans ingested it may also be necessary to determine the type of fructan ingested. The effect of the fructan may be related to for example the speed and degree of breakdown. Thus, a long chain branched chain fructan may pose less of a risk than a short straight chain fructan. Furthermore there may be large numbers of performance horses that suffer from subclinical laminitis as a result of their high grain-low forage diets.

The upper part of the equine gastrointestinal tract has a relatively small capacity and the horse has digestive and metabolic limitations to high grain, starch and sugar based diets. Large grain meals or any overload of starch or sugar may overwhelm the digestive capacity of the stomach and small intestine leading to much of the material passing through to the hindgut where it may be rapidly fermented. This fermentation leads to a production of excess lactic acid and a drop in hindgut pH. The main lactate producing bacterial genus are Streptococcus and Lactobacilli, more particularly the species S. bovis and L. mucosae, S. ruminatum and L. reuteri streptococcus infantarius ssp coli, as well as other equine hindgut streptococcal species. Production of lactic acid causes a rapid drop in the pH of the hindgut from about 7 to 6 or less. At this pH, acid resistant bacteria can thrive, potentially altering the balance of the flora, which in turn, potentially affects normal fermentation. At extremes of pH predominantly only acid resistant bacteria will persist. The increasingly acid environment affects the intestinal mucosa in particular, increasing permeability. This process can result in diarrhea, colic and absorption of substances such as endotoxin from the dead bacteria and other compounds such as vasoactive amines and various exotoxins including the matrix metalloprotease activators, believed to be involved in the pathogenesis of laminitis.

Conventional methods for the prevention and/or treatment of laminitis include changes in the feeding and/or keeping of the animals to reduce their access to carbohydrates (such as cereals, high sugar/starch grasses and other plants, high fructan grasses). Alternative treatments include administration of antibiotics to reduce the number of Gram positive bacteria in the caecum and the large intestine. Reduction of the Gram positive bacteria population should lead to a decrease in the level of lactate produced. While the administration of such antibiotics may be effective, existing antibiotics for use in the treatment of laminitis have a number of side effects which may be problematic. For example the commonly used antibiotic Virginiamycin is also classed as a growth promoter and is currently not available for general use in Europe. Furthermore, the use of antibiotics is severely compromised by emerging antibiotic resistance in bacteria.

It will be appreciated by a person skilled in the art that the causes of laminitis are both complex and multifactorial. There is therefore a need in the art for an effective multifactorial approach to the prevention of laminitis. Such an approach is provided by the composition of the present invention.

The first aspect of the present invention therefore provides a composition comprising one or more of a matrix metalloprotease inhibitor, a decarboxylase inhibitor, a fructanase enzyme and/or a flavonoid, for use in the prevention and/or treatment of laminitis.

In one feature of the first aspect, the composition comprises a matrix metalloprotease inhibitor and one or more of a decarboxylase inhibitor, a fructanase enzyme and/or a flavonoid.

In another feature, the composition comprises a decarboxylase inhibitor and one or more of a matrix metalloprotease inhibitor, a fructanase enzyme and/or a flavonoid.

In another feature, the composition comprises a fructanase enzyme and one or more of a matrix metalloprotease inhibitior, a decarboxylase inhibitor and/or a flavonoid.

In another feature, the composition comprises a flavonoid and one or more of a matrix metalloprotease inhibitior, a decarboxylase inhibitor and/or a fructanase enzyme.

In a particular feature of the first aspect, the composition comprises two or more of a matrix metalloprotease inhibitor, a decarboxylase inhibitor, a fructanase enzyme and/or a flavonoid.

In another feature of the first aspect, the composition comprises three or more of a matrix metalloprotease inhibitor, a decarboxylase inhibitor, a fructanase enzyme and/or a flavonoid.

In a further feature of the first aspect, the composition comprises a matrix metalloprotease inhibitor, a decarboxylase inhibitor, a fructanase enzyme and a flavonoid.

In another feature of the first aspect, the composition comprises a matrix metalloprotease inhibitor, a decarboxylase inhibitor and a flavonoid.

The composition of the first aspect can comprise a matrix metalloprotease inhibitor.

The matrix metalloprotease inhibitor may be provided by a natural or a synthetic source or a combination thereof. The matrix metalloprotease inhibitor can be provided as a purified, semi-purified or a crude extract. Alternatively or in addition, the matrix metalloprotease inhibitor may be provided by the addition of a natural source, such as a foodstuff.

For example, the matrix metalloprotease inhibitor for the present invention may be provided by the inclusion of ginger and/or curcumin, or a crude, semi-purified or purified extract therefrom.

For the purposes of this invention, the matrix metalloprotease inhibitor can act to prevent or reduce the activation or activity of a matrix metalloprotease. Alternatively, the matrix metalloprotease inhibitor may prevent or reduce the effect of the action of the matrix metalloprotease. The matrix metalloprotease inhibitor may act to prevent or reduce the activation or activity of a matrix metalloprotease and prevent or reduce the effect of the action of the matrix metalloprotease. Matrix metalloprotease enzymes, in particular MMP 2 and MMP 9 are believed to be involved in the aetiology of laminitis. Specifically an increase in MMP activity is believed to be involved in the degenerative cascade that results in the separation of the dermal and the epidermal laminae leading to the collapse of the equine foot.

Inhibitors such as those provided by curcumin may have activity against one or more matrix metalloprotease enzymes, for example curcumin has activity against MMP2, MMP3, MMP9 and MMP13. In addition to its inhibitory activity, curcumin has strong antiinflammatory and antioxidant properties.

The MMP inhibitor is preferably provided at a level of 0.1-120 mg per kg bodyweight per day, preferably 1-60 mg per kg bodyweight per day, more preferably 10-40 mg per kg bodyweight per day.

The composition of the first aspect of the invention can comprise a decarboxylase inhibitor.

The decarboxylase inhibitor of the first aspect of the invention can be provided by a natural source, and/or a synthetic source. The decarboxylase inhibitor may further be provided by a mixture of one or more natural sources and/or one or more synthetic sources. In particular, a portion of the decarboxylase inhibitor can be provided by a synthetic source and a portion of the decarboxylase inhibitors provided by a natural source. The decarboxylase inhibitor is preferably provided by a natural source.

The decarboxylase inhibitor can be provided by a natural source such as thyme, coriander, rosemary, mint, liquorice and berries. Alternatively, the decarboxylase inhibitor can be provided as an extract from the natural source, such as thymol (from thyme), soya isoflavones, rosemarinic acid (from rosemary and mint families), isoliquiritigenin (from liquorice), hydroxycinnamic acid (from various berries and plants). The decarboxylase inhibitor can be fully or partially isolated from the natural source. Alternatively, the decarboxylase inhibitors for the purpose of this invention can be carbidopa, benserazide, iodoacetate, isobutylamine, isopentanoate, PCMB, silver nitrate, mercury chloride, hydroxylamine, potassium cyanide, penicillamine, semicarbazide, glycine, alpha-fluoromethyl(1′4-dihydroxyphenyl)alanine, alpha-fluoromethyltyrosine, 3-indoleacetamide, 3-indolealdehyde, beta-phenylethylamine, DL-m-tyrosine, dopamine, epinephine, L-3,4-dihydroxyphenyl alanine, L-phenylalanine, L-tyrosine, Norepinephnine and/or L-tyramine.

The decarboxylase enzyme inhibited by the inhibitor of the present invention includes a bacterial decarboxylase enzyme for example a decarboxylase enzyme from a Gram-positive or a Gram-negative bacteria, more preferably a decarboxylase enzyme from Streptococci or Lactobacilli. More preferably, the decarboxylase enzyme for the present invention decarboxylases an amino acid, for example a valine decarboxylase, a leucine decarboxylase, a tyrosine decarboxylase, a phenylalanine decarboxylase or an aromatic L-amino acid decarboxylase. The decarboxylase enzyme preferably causes the decarboxylation of an amino acid to provide the corresponding amine. Preferably, the decarboxylase enzyme catalyses the decarboxylation of valine, leucine, tyrosine, phenylalanine or tryptophan. The decarboxylase inhibitor is therefore preferably a valine decarboxylase inhibitor, a leucine decarboxylase inhibitor, a tyrosine decarboxylase inhibitor, a phenylalanine decarboxylase inhibitor, a tryptophan decarboxylase inhibitor and/or an aromatic L-amino acid decarboxylase. It will be appreciated that a particular decarboxylase inhibitor may have activity against one or more decarboxylase enzymes. Alternatively, two or more inhibitors can be used in combination to inhibit the required decarboxylase enzymes.

The decarboxylase inhibitors can be competitive or non-competitive inhibitors of a decarboxylase enzyme. The activity of the decarboxylase enzyme can be completely inhibited or can be reduced to a level such that laminitis is prevented and/or treated.

The composition of the present invention is provided to prevent the onset of laminitis or reduce the likelihood of the onset of laminitis. The composition is particularly useful for those animals having or being susceptible to an overload of carbohydrate.

When horses are subjected to a carbohydrate overload a lactic acidosis develops in the hindgut as previously described above. In an attempt to regulate their intracellular pH, bacteria decarboxylate amino acids to form amines. These are actively transported out of the bacterial cell and so are found in the gut lumen. The drop in pH of the hindgut causes the gut mucosa to become permeable to many substances possibly including these amines. The substances can then travel in the bloodstream to the peripheral circulation where it is postulated that the amines may have a role in causing laminitis due to their role in promoting vasoconstriction, in particular in promoting vasoconstriction of the venous side of the circulation.

It is postulated by the inventors that the provision of a decarboxylase inhibitor will reduce the decarboxylation of amino acids by bacteria in the hindgut of an animal, thereby reducing or preventing the production of amines that can enter the bloodstream through the damaged gastrointestinal wall, reach the peripheral circulation and there have vasoconstrictive effects.

The enzyme inhibitor of the present invention is preferably acid stable and can survive the passage through the stomach to the small intestine and hind gut. Alternatively, the composition provides a targeted release of the decarboxylase inhibitor in the hindgut. Such targeting may be achieved by timed-release, pH sensitive release, provision as masked form for example an ester that can be released by the hindgut bacteria, etc. The enzyme inhibitor can be provided in an encapsulated form, attached to a solid support or cross-linked to one or more further inhibitors.

The composition of the first aspect may comprise a fructanase enzyme. Examples of a fructanase enzyme of the invention include Flurozyme, inulinase, exo-inulinase, endo-inulinase, Fructozyme or Megazyme.

The fructanase enzyme for the present invention includes any enzyme which can degrade fructan. For the purposes of this invention, degradation means the cleavage of a polysaccharide chain by the fructanase enzyme. Such degradation can occur by hydrolysis or by a transfer reaction to an acceptor molecule. Degradation of fructan can occur by exo-degradation (i.e. removal of fructose monomers progressively from one and/or both ends of a fructan molecule) or endo-degradation (i.e. cleavage of the fructan molecule at specific points in the middle of the fructan molecule to form two or more oligomers). For the purpose of the present invention, the fructanase enzyme is provided to degrade fructan in the stomach and small intestine thereby reducing the amount of fructan which reaches the hindgut. Provision of the fructanase enzyme therefore prevents and/or reduces the drop in hindgut pH and prevents and/or reduces lactic acidosis.

The fructanase enzyme of the present invention is effective and stable at a range of pH including low pH. In one embodiment of the invention, the enzyme acts in the stomach and small intestine to degrade fructan into fructose monomers or oligomers or shorter polymers. In this case, the enzyme is preferably active in the stomach i.e. at a pH of 4 or below, more preferably at a pH of 3 or below. Action of the fructanase enzyme in the stomach and small intestine will help to reduce the amount of fructan that reaches the hindgut to below the ‘danger level’ that is a predisposing factor for laminitis. The fructose monomers and/or oligomers produced by the degradation of fructan, can be safely absorbed throughout the GI tract.

Alternatively or additionally, the enzyme maybe stable, but not effective, at low pH in order to allow it to survive the passage through the stomach and into the small intestine. Such an enzyme may be active at moderate pH conditions as found in the small intestine for example pH 5 to 9, preferably pH 6 to 8, more preferably pH 7.

In a preferred embodiment, the enzyme or enzymes are active in both the stomach and the small intestine, thus preventing dangerously high amounts of fructan from reaching the hindgut.

The incorporation of a fructanase enzyme into the composition of the present invention reduces and/or assists in reducing the amount of fructan reaching the hindgut. This helps to prevent or reduce a drop in hindgut pH after the ingestion of fructan-containing foodstuffs. It will therefore be appreciated that in addition to preventing laminitis in an animal, the inclusion of a fructanase enzyme will also allow an animal to ingest a larger volume of fructan-containing food without the usual associated risk of laminitis. Providing the composition of the invention to an animal will therefore allow a more normal management regimen to be followed. A person skilled in the art would appreciate however that the consumption of fructan containing foods should be monitored and kept within acceptable levels especially for those animals susceptible or pre-disposed to laminitis. Background susceptibility is dependent on for example genetics, breed, insulin sensitivity, endothelial dysfunction etc, however the present invention will allow such horses to be kept under a more normal management regimen with a reduced risk of developing laminitis. For example a fat pony turned out to grass for an hour a day will effectively be able to eat more forage before it passes the triggering threshold for developing laminitis.

In a preferred embodiment of the invention, the fructanase enzyme is provided in a slow release formulation so that it is retained in or presented to the stomach and/or small intestine for as long as possible. This enables the enzyme to degrade a higher proportion of the fructan passing therethrough. The enzyme may be provided in a slow release formulation. Alternatively, the fructanase enzyme may be provided in combination with oil. It is postulated that the presence of oil will delay gastric emptying therefore increasing the timescale during which the fructanase enzyme is in the stomach.

The enzyme is preferably temperature stable so it can withstand manufacturing processes for example pelleting. For example the enzyme is stable at 40° C. or above, preferably 50° C. or above, more preferably 60° C. or above and can be formulated into a foodstuff without losing some or all its activity. Temperatures reached during pelleting are in the range of 50-70° C. depending on the ingredients.

The enzyme of the present invention can be provided from a commercial source (for instance as a purified or semi-purified enzyme or from a genetically modified bacteria) and/or can be obtained from a natural source. Examples of such natural sources include bacteria, protozoa, yeasts and fungi capable of degrading fructan such as Aspergillus niger, Lactobacillus paracasei, Streptococcus mutans, Actinomyces naeslundii, and/or Kluyreromyces marxianus. If the enzyme is obtained from a natural source it can be provided as an isolated enzyme, a semi-isolated enzyme or a crude extract. The enzyme can further be provided in the composition by the addition of the natural source (i.e. the micro-organism). In this case the activity of the micro-organism can be reduced or removed, for example by lysing the micro-organism. The natural source can therefore be live, lysed or denatured and can be provided in solution or dried, for example, air dried or freeze dried. Alternatively the micro-organism can be a food-safe probiotic.

The fructanase enzyme can be provided as a wild type or modified enzyme. Modification of the enzyme can be carried out by the production of a mutant gene encoding said enzyme and the expression thereof or by chemical and/or biological modification of the enzyme. Said enzyme can for example be encapsulated, attached to a solid support or crosslinked to one or more further fructanase enzymes. The fructanase enzyme is preferably provided in a slow release formulation i.e. by encapsulation.

The enzyme of the present invention preferably degrades fructan particularly grass fructan, specifically fructans present in different species of grasses. Examples of such enzymes include fructanase, inulinase, exo-inulinase and/or endo-inulinase Flurozyme, Fructozyme or Megazyme.

The composition of the first aspect can comprise a matrix metalloprotease inhibitor, decarboxylase inhibitor and a fructanase enzyme or a decarboxylase inhibitor and a fructanase enzyme. The combination of the decarboxylase inhibitor and the fructanase enzyme provides particularly efficient prevention of laminitis.

The fructanase enzyme is preferably provided at a level of from 0.01 to 100 g/kg of composition of preferably 0.1 to 10 g/kg of composition, more preferably at a level of 0.4-1.0 g/kg of composition.

The composition of the invention may comprise a flavonoid compound. For the purpose of this invention, the flavonoid compound may comprise one or more of a flavonol (for example quercitin, rutin, morin, hesperidine), a flavone (for example acacetin, apigenin, rutin, luteolin), an isoflavone (for example genistein), a flavanol (for example catechin and polymers thereof, procyanidins), a flavanon (for example taxifolin), a dihydroflavonol, an anthocyanin and/or an anthocyanidin. It will be appreciated that polyphenols are also encompassed within the term “flavonoid” for the present application. Further examples of flavonoids include daidzein, ginkgetin, tangeretin, kaempferol, myricetin, fisetin, isorhamnetin, naringenin, eriodictyol, thearubigins, tamarixetin, malvidin, peonidin, petunidin, and/or delphinidin.

The flavonoids may be provided by a natural or a synthetic source or a combination thereof. The flavonoids can be provided as a purified, semi-purified or a crude extract. The flavonoids may be provided by the addition of a natural source such as a foodstuff.

Examples of foodstuffs rich in flavanoids (such as flavanols) for the present invention include peanut skins, cinnamon including cinnamon bark, grape seed extract, pine kernal extract, apple skins, green tea including decaffeinated green tea, cocopolyphenyls, cloves, cumin, curcumin pomegranate, elderberry, prune, peach, apricot, soyisoflavones, beansprouts, miso, chickpeas and/or mint and/or extracts thereof.

Without being bound by scientific theory, it is proposed that the flavonoids act as vasodilators and therefore act to maintain blood flow to the hoof and to counteract the effect of any vasoconstricting compounds such as amines. They may also act as antioxidants in their own right, may reduce platelet aggregation, may act as NO donors and may in particular, have modulatory effects within cells which help to defend against extracellular oxidative stress.

The flavonoids may be provided at a level of 0.01-100 mg/kg bodyweight per day of active flavonoid, preferably at a level of 0.1-75 mg/kg bodyweight per day of active flavonoid, more preferably at a level of 1-50 mg/kg bodyweight per day of active flavonoid, most preferably at a level of 10 mg/kg bodyweight per day or above.

Preferably, the composition of the invention comprises two or more of the fructanase enzyme, decarboxylase inhibitor, and/or flavonoid in any combination thereof. More preferably the composition comprises a matrix metalloprotease inhibitor, a decarboxylase inhibitor and a flavonoid.

The composition of the present invention may include one or more additional enzymes. Preferably said additional enzymes hydrolyse carbohydrate within the small intestine so reducing the amount of carbohydrate that reaches the hindgut. Examples of such enzymes include alpha-galactosidase, beta-glucanase and/or pectinase.

The components of the composition for the purposes of this invention can be isolated from or provided by a natural source. The required component can be removed from the other components of the natural source and purified until only the required component is present. Alternatively, the required component may be partially purified from the other components of the natural source (and provided for example as a crude or partially purified extract of the natural source) or provided by the addition of the natural source. When the component is isolated from or provided by a natural source, the natural source can be chemically and/or biologically modified, for example by genetic modification. A synthetic source for the purposes of this invention provides a component substantially free of any other materials which may then be mixed with other components if required. The synthetic source of the component is more than 70% pure, preferably more than 85% pure, more preferably more than 95% pure prior to mixing with any other components. The synthetic source can be any material which has been prepared from available starting materials by a series of biochemical or chemical reactions. The product of these reactions can then be partially or fully purified. The structure of the component from the synthetic source may correspond exactly to the structure of the component in nature or it can be analogue of that structure. The synthetic component may be provided in a form, for example a masked form such as an ester or amide, which can be modified in the body to produce one or more active components.

The composition may require treatment with heat during its preparation. Each of the components of the composition is therefore preferably stable to heat. At least one of the components of the composition is preferably acid stable and can survive exposure to and passage through the stomach to the small intestine and/or hindgut. Alternatively the composition may provide targeted release of the components of the composition in the stomach, small intestine and/or hindgut, for example by timed release, pH sensitive release, provision of the composition and/or components of the composition in a masked form which can be released for example by hindgut bacteria. Alternatively the composition may be active in the stomach and/or small intestine and/or the hindgut.

The composition of the present invention can be provided for any ungulate animal. Examples of such animals include horses and/or ponies or other herbivores such as cows, goats and/or sheep.

The composition of the first aspect can be provided as a solid, liquid or semi-solid form. In particular, the composition can be provided as a powder, an aerosol, a bolus, a gel, drops, a solution, a syrup, a suspension, an emulsion, a tablet, or a capsule. The composition could be provided as a solid for addition to water or as an oil based liquid. The components of the composition can be mixed with any suitable carrier routinely used in the art such as magnesium stearate, starch, lactose, sucrose, microcrystalline cellulose, water, ethanol, glycerine, polyethylene glycol, an oil, an aqueous gum, cellulose, silicate, tragacanth, gelatin, glycerine, etc. The composition may additionally comprise a suspending agent, a preservative, a flavouring or a colouring agent.

The composition may be administered by any conventional method for example by oral (including inhalation), parenteral, mucosal (i.e. buccol, sublingual, nasal), rectal or transdermal administration and the compositions adapted accordingly.

The composition of the first aspect can be provided as a food supplement or a foodstuff. The components of the composition may be added directly to a food supplement or a foodstuff. Alternatively, the components may be added to a carrier which can then be added to a food supplement or a foodstuff prior to administration to an animal.

The food supplement or foodstuff of the first aspect of the invention can be solid, semi-solid, or liquid for example the food supplement or foodstuff could be in the form of a powder, a pellet or a drink. The food supplement or foodstuff can additionally contain ingredients which enable the foodstuff to be formulated in a particular form. For example the foodstuff can contain molasses or a molasses/oil mixture for example cane molasses with approximately 6% or above oil such as Molglo (e.g. to bind the ingredients together or as a palatability agent) or oat feed, wheat feed or another suitable filler ingredient. The foodstuff may also contain fibre sources such as grasses, straw (chopped or ground), sugar beet, soya hulls and oats, and fat sources such as rice bran, corn oil, soya oil, processed canola oil, coconut oil, linseed (flaxseed), palm oil or sunflower oil.

The food supplement can be added to a food or administered prior to or after feeding. The supplement could be administered together with a standard foodstuff used or with the foodstuff of the invention. The mixing may occur when the foodstuff is prepared or packaged or may occur when the foodstuff is provided to the animals. The supplement may alternatively be supplied as a topping to a foodstuff. The food supplement can be in the form of a food snack or a drink (for example snack bars, biscuits and sweet products). The drink may be aqueous or oil based.

The foodstuff or food supplement of the present invention may be provided as a solid block which can be provided in a field or in a stable, or a feeding system operated by the animal.

The composition of the first aspect of the invention can be provided in varying quantities per day. The composition could be provided as a slow release formulation. Such a slow release formulation could allow the composition to be provided once or twice a day. The slow release formulation could allow release of the composition at any stage in the gastrointestinal tract from mouth to colon. Alternatively, the preparation could be available to the animal continuously. Such a composition could for example be presented in a lick formulation together with other palatable ingredients. The formulation could then be left on pasture and the animal could voluntarily periodically ingest the preparation. The composition could be freely available throughout the day to allow the animals to eat or ingest at will. Alternatively, the composition is provided once or twice a day, i.e. when the animals are fed, returned to stable etc.

The composition of the present invention can be provided to an animal for example a horse or pony. Preferably the composition is provided to a horse which is kept out at grass/pasture (e.g. fat ponies), or is fed a high cereal diet, or has a degree of insulin resistance through dietary management, or has periods of inactivity, or has inflammation or is stabled. The composition can be used to prevent acute laminitis, to reduce the likelihood of acute laminitis or is provided for an animal which is susceptible to or suffering from chronic laminitis. Where the foodstuff is provided for an animal suffering from chronic laminitis, the foodstuff is provided as a preventative treatment or can be provided in combination with a conventional therapy as an adjunct therapy. Where an animal is identified as being susceptible to laminitis, the composition can be provided as a preventative therapy or can be provided in combination with a conventional therapy as an adjunct therapy. The composition is therefore particularly provided to animals deemed at risk of developing laminitis.

For horses who are not suffering from laminitis and who are not deemed susceptible thereto, the composition can be used to prevent laminitis. In particular, the composition is used to reduce the risk or likelihood of laminitis occurring in an animal. The composition can be used to reduce the susceptibility of an animal to laminitis and remove the animal from the “danger zone” wherein laminitis can occur.

Laminitis is recognised in practice by the development of lameness or hoof abnormality following a physical or biological insult (such as carbohydrate overload, physical injury etc). It will be appreciated that there is a time delay between the insult and the identifiable onset of lameness.

The composition of the present invention is provided for the prevention of laminitis. The use of the composition can be termed as “medicine” although this term does not necessarily mean that the composition is a licensed medicament subject to regulatory authorisation requirements. The composition of the present application may be used in medicine.

The term “prevention” according to the first aspect of the invention relates to the prevention, delay or reduction of the clinical signs of laminitis. It will be appreciated by a person skilled in the art that the development of laminitis involves a biological trigger (i.e. ingestion of carbohydrate) which produces a biological response (i.e. drop in hindgut pH, production of vasoactive amines) which leads to one or more clinical symptoms (i.e. lameness, increase in foot/hoof temperature, inflammation of the foot and/or hoof, detection of digital pulse, abnormal posture, abnormal gait, abnormal distribution of weight).

The composition of the first aspect of the invention can reduce, delay or prevent the biological response and/or reduce, delay or prevent the production of a clinical symptom by the administration of the composition of the invention. The term “prevention” encompasses “assisting in the prevention” of laminitis, for example as an adjunct therapy. The composition may prevent laminitis by acting in the hoof, the fore-gut and/or the hindgut or any other region of the body that is affected.

The composition of the present invention can be used as adjunct therapy. The composition can therefore be provided with a conventional therapy in order to prevent or reduce the clinical symptoms suffered by an animal. The use of the composition may allow the reliance on drug therapy to be reduced. Alternatively, the animal may exhibit less symptoms or the severity of the symptoms may be reduced.

The presence of the components and the relative quantities thereof will depend on the physiological state of the animal. For example, horses used for competition who are housed in stables could be provided with a foodstuff comprising a matrix metalloprotease inhibitor, flavonoid and a decarboxylase inhibitor. Alternatively, animals kept on grass or fed on high fructan containing hay or other foliage could be provided with a foodstuff comprising a fructanase enzyme. It should be appreciated however, that animals kept on grass or fed on high fructan containing hay or other foliage could be provided with a foodstuff comprising one or more of a matrix metalloprotease inhibitor, flavonoid and a decarboxylase inhibitor.

The composition of the first aspect of the invention may comprise other components which aid in the prevention of laminitis. For example, the composition of the first aspect may additionally comprise one or more of an antioxidant, a nitric oxide donor, a hindgut buffer, a full gut buffer, an insulin promoter, an inhibitor of insulin resistance, or an agent to decrease the passage and/or availability of fructan.

Examples of antioxidants for the purposes of the present invention include vitamin A, vitamin C, vitamin E, selenium, carotenoid, flavonoid, phyto-estrogen, proanthrocyanidins, and/or ubiquinone. The presence of antioxidant compounds may prevent or reduce the effect of free radical damage in the hoofs.

The composition of this invention may further comprise nitric oxide donors such as arginine, or an agent which supports or could support endothelial function. As previous discussed, a flavonoid may act as a nitric oxide donor and can be provided to have this function for the purposes of the present invention.

For the purposes of this invention, the composition may comprise a hindgut buffer such as magnesium oxide. The hindgut buffer acts to prevent and/or reduce the drop in hindgut pH thereby preventing and/or reducing lactic acidosis. The hindgut buffer may act to increase the pH of the hindgut. The buffer should be provided in a form that it can get to the hindgut, for example it may be encapsulated so that it releases selectively in the hindgut.

Finally, the composition may comprise one or more insulin promoters. The provision of an insulin promoter prevents the reduction of glucose entry into the lamellar basal cells of the hoof and or reduces the degree of insulin resistance thereby lowering the circulating glucose and/or insulin levels and/or reducing fluctuations in circulating glucose and/or insulin levels. Sources of insulin promoters include cinnamon, bayleaves, tumeric, witch hazel, black tea, green tea, all spice, nutmeg, mushrooms, brewers yeast, Korean ginseng, flaxseed meal and/or basil.

The composition of the present invention preferably comprises a decarboxylase inhibitor and a flavonoid. The composition may further comprise an antioxidant, a hindgut buffer and/or an insulin promoter.

The second aspect of the invention provides the use of one or more of a matrix metalloprotease inhibitor, a decarboxylase inhibitor, a fructanase enzyme and/or a flavonoid in the manufacture of a composition for the prevention of laminitis. Preferably, the composition comprises a matrix metalloprotease inhibitor, a decarboxylase enzyme, a flavonoid and a fructanase enzyme. Alternatively, the composition comprises a matrix metalloprotease inhibitor, a decarboxylase enzyme and a flavonoid.

The third aspect of the invention provides a method of preventing laminitis comprising administering one or more of a matrix metalloprotease inhibitor, a decarboxylase inhibitor, a fructanase enzyme and/or a flavonoid. Preferably, the method comprises administering a matrix metalloprotease inhibitor, a decarboxylase inhibitor, a flavonoid and a fructanase enzyme. Alternatively, the method comprises administering a matrix metalloprotease inhibitor, a decarboxylase enzyme and a flavonoid.

The method comprises administering the composition, daily, as needed throughout the day, twice daily or continuously throughout the day. The composition can be administered throughout the year, or during periods of time at which the risk of laminitis is increased (i.e. at periods wherein levels of fructan in grass is high i.e. spring and/or autumn).

The fourth aspect of the invention comprises a composition comprising one or more of a matrix metalloprotease inhibitor, a decarboxylase inhibitor, a fructanase enzyme and/or a flavonoid. The composition preferably comprises a matrix metalloprotease inhibitor and/or a decarboxylase inhibitor.

More preferably, the composition comprises a matrix metalloprotease inhibitor, a decarboxylase inhibitor and a fructanase enzyme or a flavonoid. Most preferably, the composition comprises a matrix metalloprotease inhibitor, a decarboxylase inhibitor, a fructanase enzyme and a flavonoid.

The fifth aspect of the invention provides a process for the preparation of a composition as defined in the first, second and fourth aspect of the invention. The composition is preferably a foodstuff or food supplement. A foodstuff or food supplement can be prepared using conventional techniques.

In one embodiment, the food supplement is provided as a pellet. The ingredients of the food supplement are mixed together and the product mix is passed through a dye plate containing several holes of a nominated size (for example from 4 mm to 16 mm, more preferably from 4 mm to 9 mm). A minimal heat is supplied to the mixture prior its entry into the mixture to raise the temperature of the mixture to approximately 50° C. Temperatures of 95° C. have been used to raise the temperature of the mixture to the required temperature.

The foodstuff can be provided as an extruded pellet. In this process, the mixture enters the extruder where it is heated. By forcing the product along the barrel of the extruder, through a series of precision restrictors, the product is exposed to high temperatures and high pressures. The product emerges from the extruder through a shape dye where it expands as the pressure immediately returns to normal atmospheric pressure. The extruded material is then cut to the required length.

All preferred features of each of the aspects of the invention apply to all other aspects mutatis mutandis.

The invention will now be described with reference to the following non-limiting examples.

### EXAMPLES

Several species of bacteria have been identified that are known to decarboxylase amino acids in the hind-gut of horses (table 1).

TABLE 1
Non exhaustive list of bacteria in the horses
hindgut that decarboxylase amino acids

Streptococcus bovis
Lactobacillus mucosae
Lactobacillus reuteri
Lactobacillus salivarius
Lactobacillus delbruekii
Lactobacillus fermentum

### Amine Production and pH Changes in a Model of Carbohydrate Overload

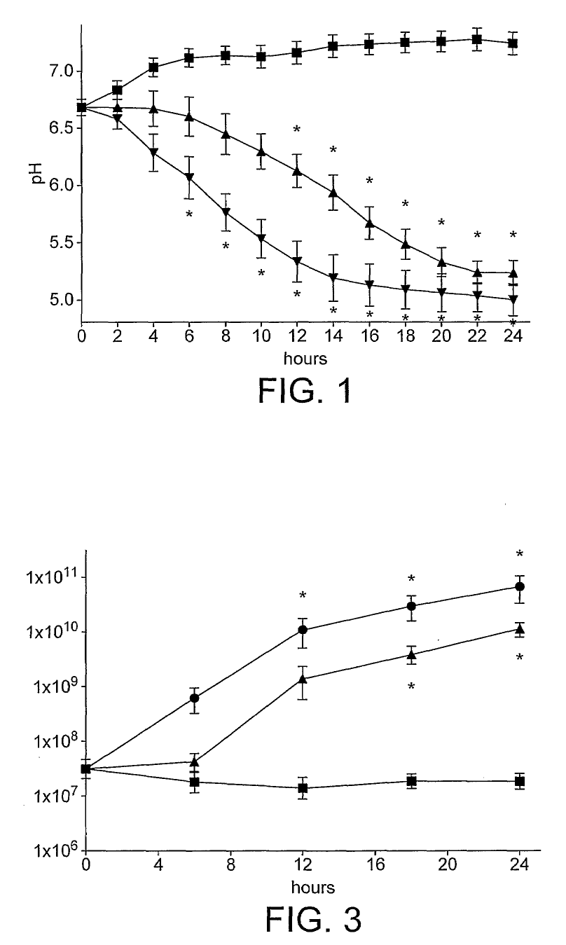

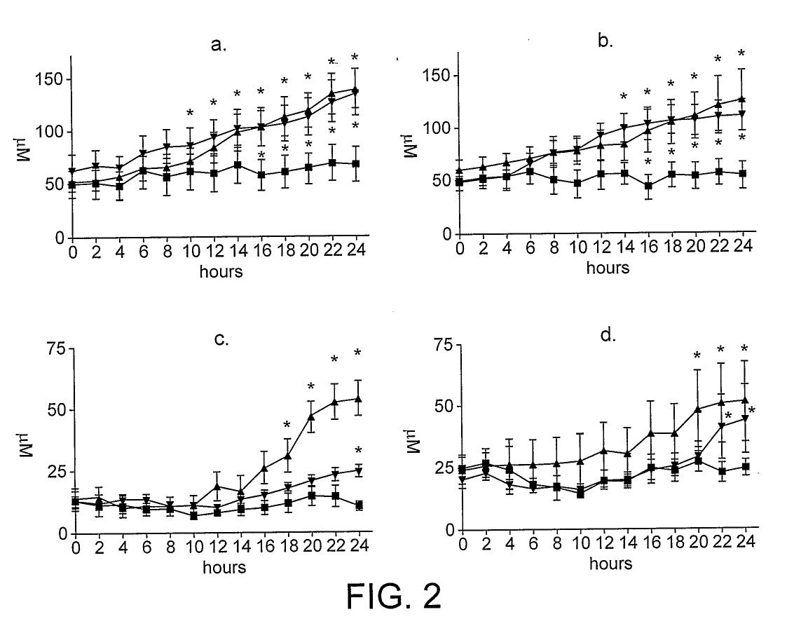

Work was carried out using an in vitro model of an equine hindgut to show:
pH changes in the equine hindgut cause by a simulated carbohydrate overload (results shown in FIG. 1)
the effects of carbohydrate overload on the amine production by hindgut bacteria (results shown in FIG. 2)

When incubated with cornstarch or inulin (commercially available fructan) the pH fell significantly in the model system over a 24 hour period, compared with a control that was incubated without added carbohydrate (see FIG. 1). Amine production was also measured over the same timescale in the in vitro model system, where cecal contents were incubated with cornstarch, inulin or without added carbohydrate. At the end of a 24 hour period there was significantly greater concentrations of the caecal-derived amines phenylethylamine, isoamylamine, putriscine and cadaverine in the culture vessel (see FIG. 2) when the incubation included a carbohydrate source compared to the control.

### pH Changes in Model Equine Hindgut (FIG. 1)

Effects of carbohydrate overload on pH of equine cecal contents incubated anaerobically in vitro. Cecal contents were divided into aliquots and incubated for 24 h with the inclusion of either inulin (1 g/100 mL; [symbol] corn starch (1 g/100 mL; ▴) or without added carbohydrate (control; ▪). Each value set out in FIG. 1 represents the mean±s.e.m. of estimates taken from 10 separate experiments.

* indicates significant difference compared with control values, twoway repeated measures analysis of variance with Bonferroni's post hoc test.

### Amine Production in an Equine Hindgut Model (FIG. 2)

Effects of carbohydrate overload on amine concentrations in equine cecal contents incubated anaerobically in vitro. The concentrations of (a) phenylethylamine, (b) isoamylamine, (c) putrescine and (d) cadaverine were measured by high performance liquid chromatography. Cecal contents were divided into aliquots and incubated for 24 h with the inclusion of either inulin (1 g/100 mL; ▾), corn starch (1 g/100 mL; ▴) or without added carbohydrate (control; ▪). Each value set out in FIG. 2 represents the mean±s.e.m. of estimates taken from 4 separate experiments.

* indicates significant difference compared with control values, twoway repeated measures analysis of variance with Bonferroni's post hoc test.

Use of an in vitro model of carbohydrate overload to study the change in populations of caecal streptococci and lactobacilli and to establish whether certain species of these bacteria were capable of producing vasoactive amines from amino acids.

Caecal contents from 10 horses were divided into aliquots and incubated anaerobically with either corn starch or inulin (fructan; both 1 g 100 ml). Samples were taken at 6 h intervals over a 24 h period for enumeration of streptococci, lactobacilli and Gram-negative anaerobes by a dilution method onto standard selective growth media. The effects of the antibiotic virginiamycin (1 mg/100 ml), and calcium hydrogen-phosphate (CaBPO₄; 0.3 g/100 ml) were also examined. Fermentation of excess carbohydrate was associated with increases in numbers of streptococci and lactobacilli (2-3.5 log unit increases; abrogated by virginiamycin) but numbers of Gram-negative anaerobes were not significantly affected. A screening agar technique followed by 16S ribosomal DNA analysis enabled the identification of 26 different bacterial strains capable of producing one or more vasoactive amines. These included members of the species Streptococcus bovis and five different Lactobacillus spp. These data suggest that certain bacteria, whose overgrowth is associated with carbohydrate fermentation, are capable of producing vasoactive amines which may play a role in the pathogenesis of acute laminitis.

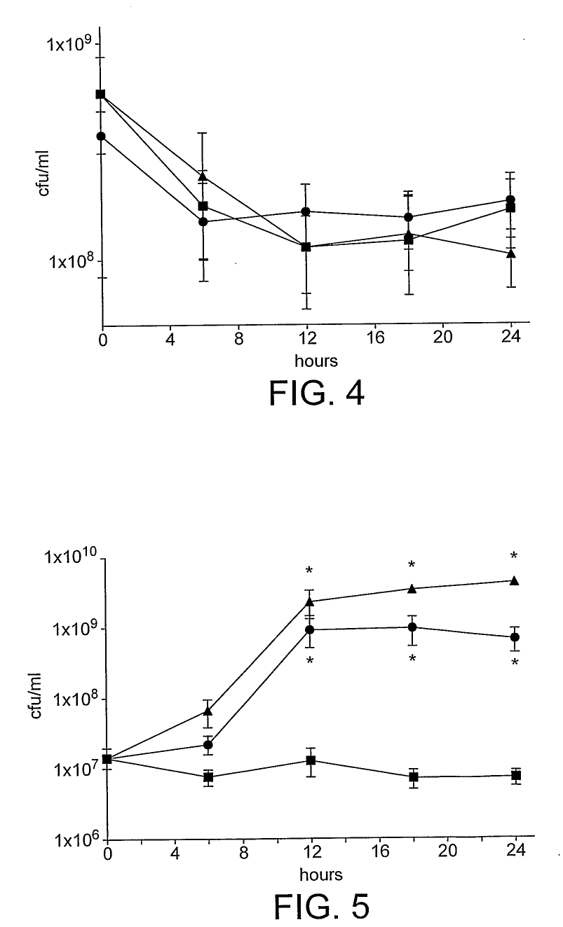

Results are set out in figures FIGS. 3, 4 and 5.

Effects of carbohydrate overload on numbers of (a) streptococci, (b) lactobacilli and (c) Gram-negative anaerobes in equine caecal contents incubated anaerobically in vitro. Caecal contents were divided into aliquots and incubated for 24 h with the inclusion of either inulin (1 g/100 ml; •), corn starch (1 g/100 ml; ▴) or without added carbohydrate (control; ▪). Bacterial numbers in each aliquot at 6 h intervals were determined by serial dilution of caecal contents in sterile RPS plated onto selective growth medium. Each value represents the geometric mean±s.e.m. of estimates taken from 10 separate experiments.

* significant difference compared with control values, twoway repeated measures analysis of variance with Bonferroni's post hoc test.

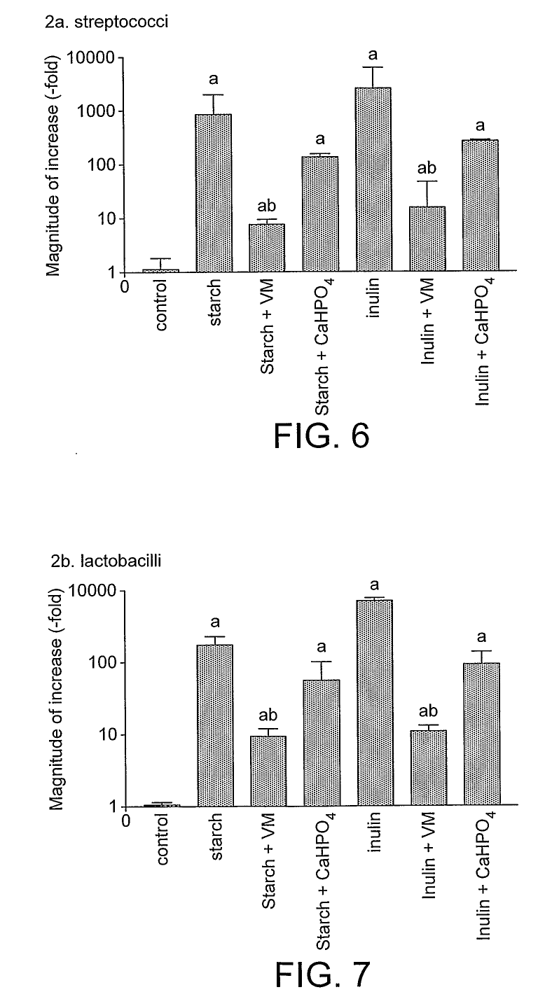

### FIGS. 6 and 7

Effects of virginiamycin and calcium hydrogen-phosphate on numbers of Streptococcus and Lactobacillus spp. in equine caecal contents induced by incubation with starch or inulin. Aliquots of caecal contents were incubated with carbohydrate in the presence or absence of virginiamycin (VM; 1 mg/100 ml) or calcium hydrogen phosphate (CaHPO₄; 30 mg/100 ml). Bacterial numbers were measured at 0 and 24 h, expressed as CFU/ml, and the magnitude of the change over this period was calculated by dividing the number of bacteria present at 24 h by the initial figure (geometric mean±s.e.m. of estimates taken from 4 separate experiments).

^{a} significant differences compared with control,
^{b} significant difference compared to starch or inulin alone, oneway analysis of variance with Fisher's multiple comparison.

### Differences in Carbohydrate in Grass at Different Times of the Year

The differences seen at different times of the growing season for pasture grass (ie winter and spring) are due to the widely varying levels of carbohydrate found in different grass varieties and at different times of the year. Work was carried out to determine the levels water soluble carbohydrate and high molecular weight carbohydrate in different pasture grasses (table 2).

TABLE 2
Different pasture grasses have different levels of
fructans on a dry matter (DM) basis of whole grass

| | Total Water Soluble | Fructan (High MW |
|---|---|---|
| Grass Variety | Carbohydrate (CHO) % DM | CHO) % DM |
| Cocksfoot | 21.3 | 11.0 |
| Timothy | 16.8 | 10.3 |
| Meadow fescue | 29.7 | 15.6 |
| Italian ryegrass | 23.0 | 7.2 |
| Perennial ryegrass | 26.6 | 10.1 |
| Hybrid ryegrass | 22.7 | 6.4 |

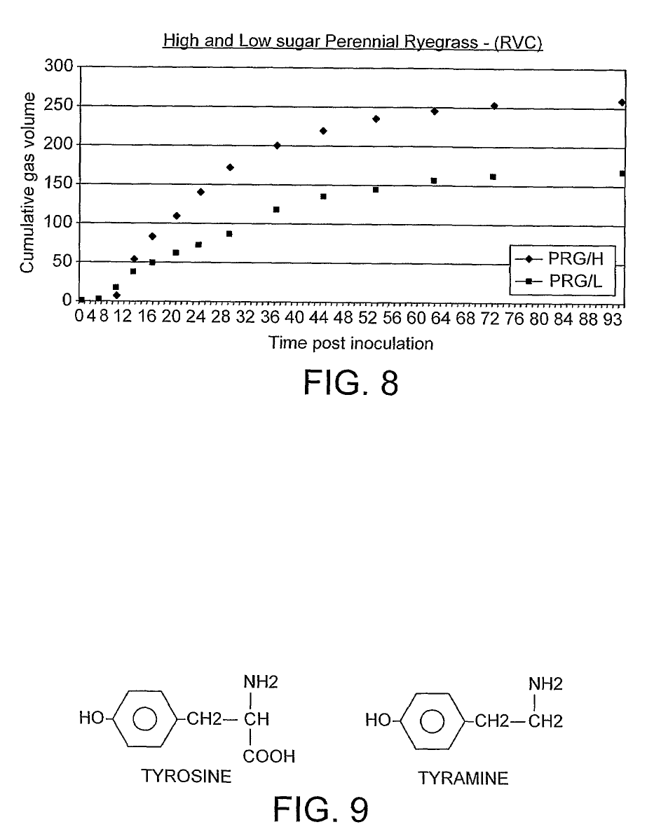

Work was carried out to show that grasses with different levels of high molecular weight carbohydrates are fermented at different rates in a model system of the equine hind gut. These results are indicated in FIG. 8.

### Activity of Fructanase Enzymes at Varying pH

Work was carried out using commercial enzymes from Sigma, Fluka and Megazyme against commercial inulin from chicory root. The optimum pH for all three enzymes was at pH 5, but there was still over 50% relative activity pH 3 and pH 6.5. The concentration of the enzyme preparations was determined by gel electrophoresis. The Megazyme proved to be the most concentrated preparation. The activity of the three enzymes was tested against chicory inulin at pH 5. Megazyme was again the most effective enzyme.

Work was carried out to show that the Megazyme enzyme (the most effective commercially available enzyme) was active against grass fructans (table 3).

TABLE 3
The activity of Megazyme against inulin, crude cocksfoot grass extract
and semi-purified soluble cocksfoot grass fructan fraction.

| Sample | Activity (U/ml) | Relative activity (%) |
|---|---|---|
| Inulin | 0.0894 | 100 |
| Crude extract | 0.0005 | 0.5 |
| Soluble fraction | 0.0094 | 10 |

Fructanase Activity from Bacterial Culture

Work was carried out to show that a bacterial culture had fructanase activity on both commercial inulin and grass fructan at a pH set to be equivalent to the range likely to be found in a horses stomach. Lactobacilli paracasei was cultured in a broth using inulin or grass fructan (0.4% w/v) as carbon source at 30° C. for 4 days. The fructanase activity of the cell suspensions (grown in inulin or grass fructan as a carbon source) on grass fructan (5 mg/ml—from cocksfoot grass) at pH 3.5 and 5.5 was measured (table 4).

TABLE 4
The activity (U/ml) of Lactobacillus Paracasei Cell suspensions
from Inulin and Grass fructan at pHs 3.5 and 5.5

| | |
|---|---|
| Cell suspension - | Cell suspension - |
| Inulin | Grass fructan |

| | | | | |
|---|---|---|---|---|
| pH | 3.5 | 5.5 | 3.5 | 5.5 |
| Activity* (U/ml) | 0.0114 | 0.056 | 0.015 | 0.022 |

*A Unit is defined as amount of enzyme that produced 1 micromole of reducing sugar (as fructose) per minute under the defined conditions.

### Amino Acid Decarboxylation

Amino acids are decarboxylased to amines in the process illustrated in FIG. 9.

### Inhibition of Decarboxylase Activity

Tyrosine decarboxylase (TDC) decarboxylases tyrosine to tyramine. Work has been carried out to show that production of tyramine is inhibited by the decarboxylase inhibitors, carbidopa and benserazide.

### Effect of Dietary Fructan Carbohydrates on Normal and Laminitic Ponies

Eleven adult, native breed ponies were used in the diet study; 5 normals and 6 predisposed to laminitis (asymptomatic at the time of the study). A basal hay diet was fed for 2 weeks, prior to the inclusion of inulin (sourced from chicory root; 3 g/kg body weight per day), fed in 3 equal feeds. The daily forage ration also included ⅔ hay, by weight, and ⅓ Readigrass® (to act as a source of amino acids). Fecal and blood samples were taken during both diets to measure amine concentration (by HPLC and LC-MS, respectively) and fecal pH.

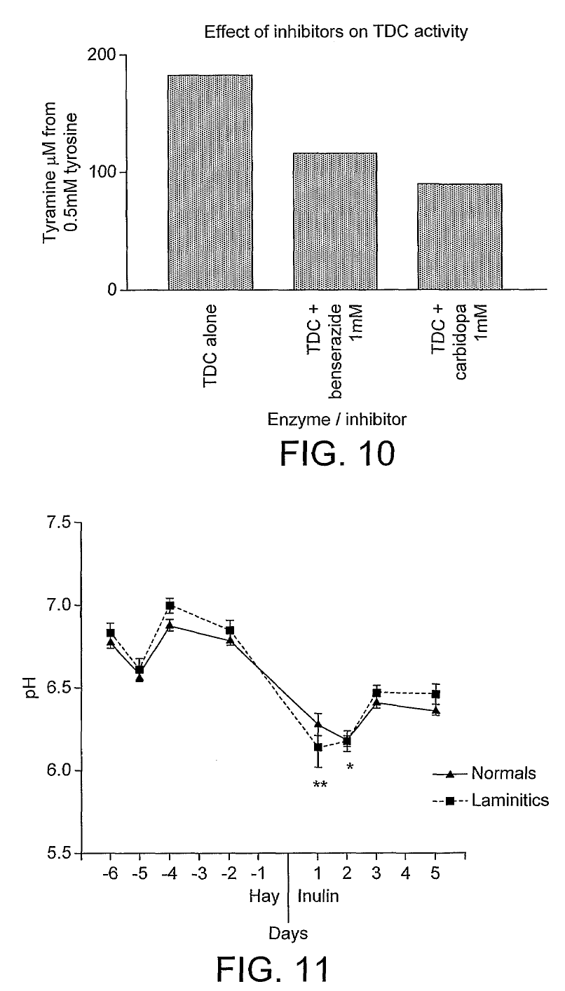

No animal showed any signs of laminitis throughout the study. The faecal pH of both normal and laminitic ponies decreased significantly by 24 hours following the beginning of the inulin diet as illustrated in FIG. 11 (mean 6.9±0.1 to 6.2±0.1; p<0.05).

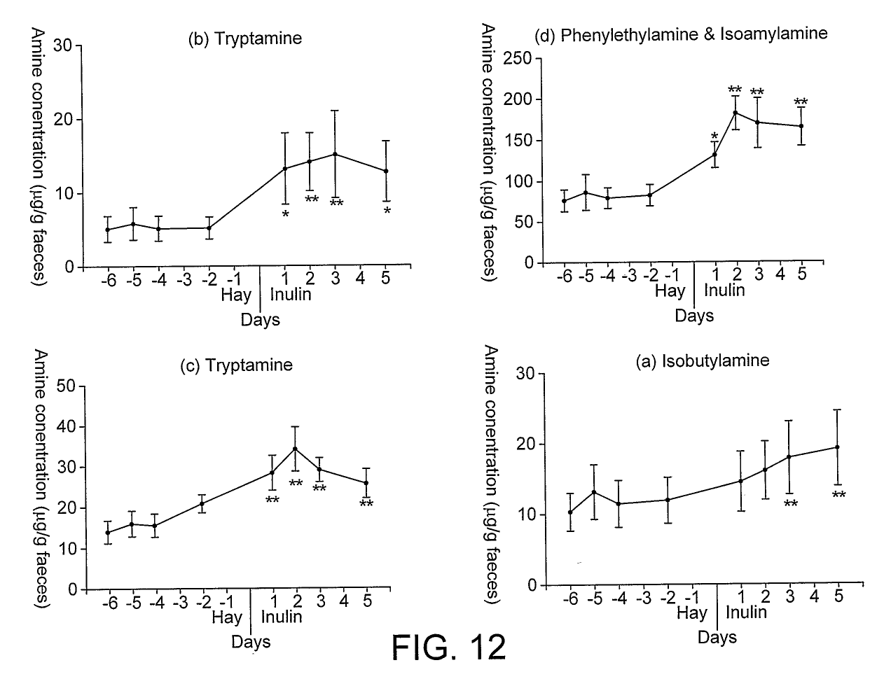

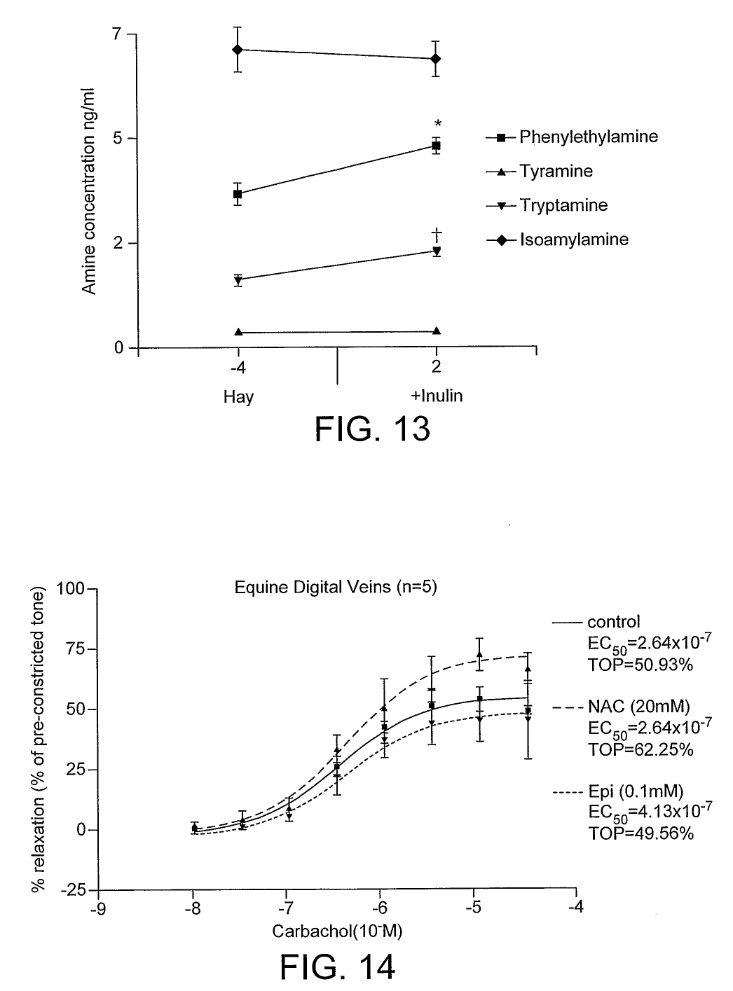

The concentration of several vasoactive amines increased significantly on the inulin diet, including tryptamine and tyramine as illustrated in FIG. 12 (58.6 and 23.3% increases, respectively). Plasma concentrations of the vasoactive amines also increased as illustrated in FIG. 13 (in which *=significant increase in plasma phenylethylamine concentration (Wilcoxon signed rank test; P=0.034) and †=P=0.07 positive trend for Tryptamine).

These data demonstrate that the production of vasoactive amine compounds increases significantly in response to dietary fructan carbohydrates in vivo. This study was designed not to cause laminitis per se and changes in the pH in the large intestine were not as large as may be needed to cause significantly increased permeability of the mucosa, which would have resulted in more marked release of amines into the circulation and possible clinical signs of laminitis.

### Isolated Blood Vessel Work

The coronary band veins and the distal artery were dissected from equine hind limbs obtained from abattoir collections (animals euthanased for non related reasons), cut into rings of approximately 3 mm in length and prepared for isometric tension recording—a method established in the laboratory for looking at endothelium intact responses of equine digital. Studies looking at the effect of adding an antioxidant (eg NAC N-acetyl cysteine) to the medium of isolated digital blood vessels have shown that it can increase the relaxation above that of the control (as illustrated in FIG. 14 which shows that the antioxidant effect of NAC (scavenging of reactive oxidative species ROS) increases the relaxation of the digital blood vessels (enhances endothelium-dependant relaxation—more NO availability).

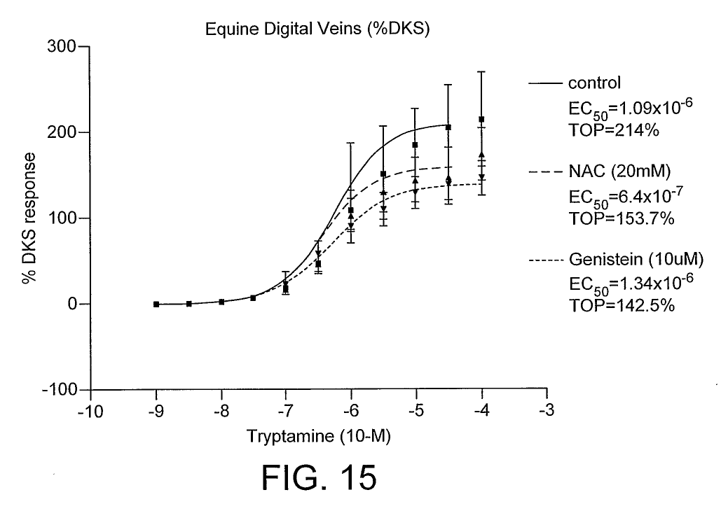

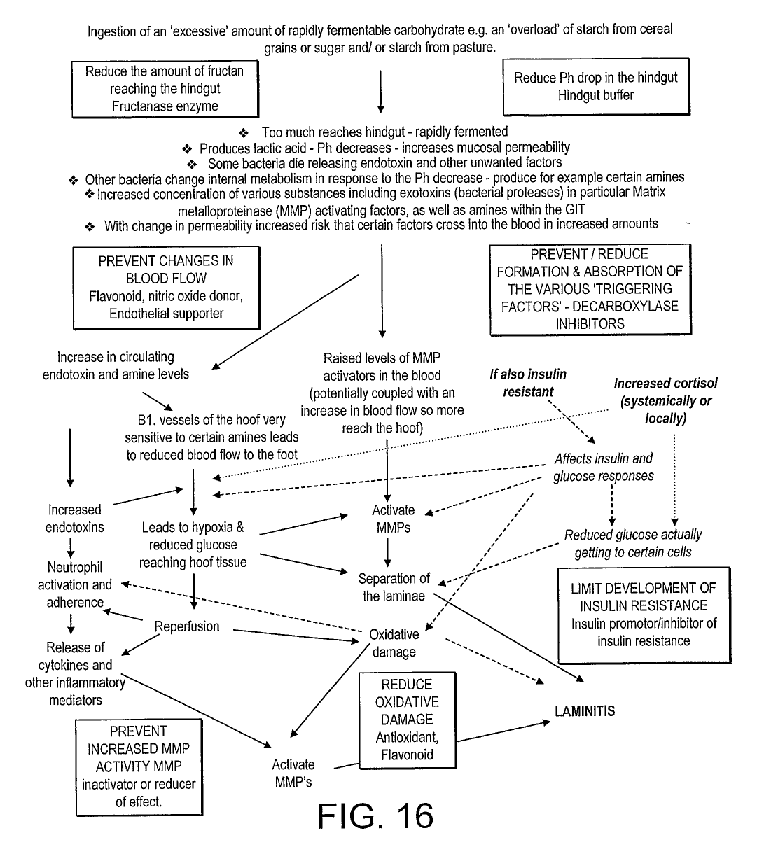

Pre incubation with an antioxidant (NAC) within the medium of isolated digital blood vessels resulted in a reduced contraction in response to the addition of these vasoactive amines on the digital veins (vasoconstriction of the venous side of the digital vasculature appears to be an important step in the pathogenesis of laminitis). This effect is even more marked when a flavonoid (genestin) is added with a reduction of 30% (as illustrated in FIG. 15 which shows that there is a reduced response to Tryptamine when NAC and especially Genisten are added to the culture medium.)

### Example of a Flavonoid of the Invention

| | Soy Isoflavones 40.0% |
|---|---|
| Botanical Name | Glycine max (L.) Merr |
| Botanical Part Used | Seeds |
| Appearance | Fine Powder |
| Colour | Light Brown Yellow |
| Odour | Characteristic |
| Taste | Characteristic |
| Assay (Total Isoflavones) | Min 40.0% |
| Genistin + Genistein | Min 21% |
| Daidzin + Daidzein | Min 19% |
| Glycitin + Glycitein | Min 1% |
| Ash | Max 5.0% |
| Bulk Density | 0.40-0.60 g/ml |

1. A composition comprising two or more of a matrix metalloprotease inhibitor, a decarboxylase inhibitor and/or a flavonoid.

2. A composition as claimed in claim 1 wherein said composition comprises a matrix metalloprotease inhibitor.

3. A composition as claimed in claim 2 wherein the matrix metalloprotease inhibitor is provided by a natural source, a synthetic source or a mixture thereof.

4. A composition as claimed in claim 3 wherein the matrix metalloprotease inhibitor is provided by ginger and/or curcumin.

5. The composition as claimed in claim 2 wherein said composition comprises a decarboxylase inhibitor.

6. The composition as claimed in claim 5 wherein the decarboxylase inhibitor is provided by a natural source, a synthetic source, or a mixture thereof

7. The composition as claimed in claim 6 wherein the decarboxylase inhibitor is one or more of iodoacetate, isobutylamine, isopentanoate, PCMB, silver nitrate, mercury chloride, hydroxylamine, potassium cyanide, penicillamine, semicarbazide, glycine, alpha-fluoromethyl(r4-dihydroxyphenyl)alanine, alpha-fluoromethyltyrosine, 3-indoleacetamide, 3-indolealdehyde, beta-phenylethylamine, DL-m-tyrosine, dopamine, epinephine, L-3,4-dihydroxyphenyl alanine, L-phenylalanine, L-tyrosine, Norepinephnine, carbidopa, benserazide and/or L-tyramine.

8. The composition as claimed in claim 7 wherein the decarboxylase inhibitor is provided by one or more of thyme, coriander and/or soya isoflavones.

9. The composition as claimed in claim 8 wherein the carboxylase inhibitor is provided as an encapsulated form, attached to a solid support or cross-linked to one or more further inhibitors.

10. (canceled)

11. (canceled)

12. (canceled)

13. (canceled)

14. (canceled)

15. The composition as claimed in claim 1 comprising a flavanoid.

16. The composition as claimed in claim 15 wherein the flavanoid is a flavanol, a flavanol, a flavone, an isoflavone and/or a flavanon.

17. The composition as claimed in claim 16 wherein the flavonoid is provided by one or more of peanut skins, cinnamon, grape seed extract, pine kernal extract, apple skins, green tea, pomegranate, elderberry, prune, peach, apricot, soyisoflavone, bean sprouts, miso, chickpeas and/or cocopolyphenyls, and/or extracts thereof.

18. The composition as claimed in any one of claim 17 wherein the flavonoid is provided at a level of 0.01-100 mg/kg body weight per day of active flavonoid.

19. The composition as claimed in any one of claim 18 further comprising one or more of alpha-galactosidase, beta-glucanase and/or pectinase.

20. The composition as claimed in any one of claim 19 further comprising one or more of an antioxidant, a nitric oxide donor, a hindgut buffer or an insulin promoter.

21. A method of manufacturing of a composition for reducing the likelihood of laminitis occurring in an animal comprising the step of incorporating one or more of a matrix metalloprotease inhibitor, a decarboxylase inhibitor and/or a flavonoid into a composition.

22. The method as claimed in claim 21 wherein a matrix metalloprotease inhibitor, a decarboxylase inhibitor and/or a flavonoid are added to the composition.

23. The method as claimed in claim 22 wherein a matrix metalloprotease inhibitor, a decarboxylase enzyme and a flavonoid are added to the composition.

24. A method of reducing the likelihood of laminitis occurring in an animal comprising the step of administering one or more of a matrix metalloprotease inhibitor, a decarboxylase inhibitor, and/or a flavonoid to an animal in need thereof.

25. The method as claimed in claim 24 comprising administering 2 or more of a matrix metalloprotease inhibitor, a decarboxylase inhibitor and/or a flavonoid.

26. The method as claimed in claim 25 comprising administering a matrix metalloprotease inhibitor, a decarboxylase inhibitor and a flavonoid.

27. The method as claimed in claim 26 wherein the animal is equine.

28-33. (canceled)
